(19) United States
(12) Patent Application Publication
(10) Pub. No.: US 2025/0199006 A1
(43) Pub. Date: Jun. 19, 2025

(54) METHOD FOR DIAGNOSING CANCER

(71) Applicants: FUNDACIÓ INSTITUT MAR D'INVESTIGACIONS MÈDIQUES (IMIM), BARCELONA (ES); UNIVERSITAT DE GIRONA, GIRONA (ES); FUNDACIÓ INSTITUT D'INVESTIGACIÓ BIOMÈDICA DE GIRONA DR. JOSEP TRUETA, SALT (ES); INSTITUT CATALÀ D'ONCOLOGIA, HOSPITALET DE LLOBREGAT (ES)

(72) Inventors: OSCAR JUAN POZO MENDOZA, SABADELL (ES); ÀLEX GÓMEZ GÓMEZ, BARCELONA (ES); GABRIEL GIL GÓMEZ, BARCELONA (ES); MANUEL RAMÓN PERA ROMAN, BARCELONA (ES); LAURA VISA TURMO, BARCELONA (ES); EDURNE ARRIOLA APERRIBAY, BARCELONA (ES); ROSA PERACAULA MIRÓ, GIRONA (ES); ESTHER LLOP ESCORIHUELA, GIRONA (ES); ERNESTO CASTRO GUTIÉRREZ, BARCELONA (ES); ADELAIDA GARCÍA VELASCO, GIRONA (ES); ESTHER FORT MARTORELL, SALT (ES); DUNIA PÉREZ DEL CAMPO, GIRONA (ES)

(21) Appl. No.: 18/843,052

(22) PCT Filed: Mar. 13, 2023
(86) PCT No.: PCT/EP2023/056335

§ 371 Date: Aug. 30, 2024

(30) Foreign Application Priority Data
Mar. 14, 2022 (EP) 22382244.6

(51) Int. Cl. G01N 33/574

(52) U.S. Cl. CPC G01N 33/57488; G01N 33/574; G01N 2570/00

(57) ABSTRACT

The combined use of biomarkers for cancer diagnosis, and in particular, the combined use of (a) 3-oxopropanoate and at least one of (b) a ketone body selected from beta-hydroxybutyrate, acetoacetate and combinations thereof, or (c) a medium chain fatty acid selected from octanoate, decanoate and combinations thereof, as a biomarker for the diagnosis of cancer. An in vitro method for diagnosing cancer in a subject based in the particular biomarker combination.

This application claims the benefit of European Patent Application no. EP22382244.6 filed Mar. 14, 2022.

### TECHNICAL FIELD

The application refers to the field of cancer diagnosis and, in particular, to biomarkers for diagnosing cancer.

### BACKGROUND ART

Cancer is one of the most devastating diseases worldwide. Pancreatic cancer, in particular, has become the third leading cause of cancer-related death in both sexes in developed countries (Siegel R L, et al, CA Cancer J Clin. 2022 January; 72 (1): 7-33). Its incidence is increasing, with the projection to be the second-leading cause of cancer death by 2030 in the US (Rahib L, et al, Cancer Research. 2014 June; 74 (11): 2913-2921).

The majority of the diagnosed pancreatic tumors (90%) are ductal adenocarcinomas (PDAC), which is one of the most lethal cancers, with a 5-year survival of less than 10% (Park W, et al, JAMA-Journal of the American Medical Association. 2021 September; 326 (9): 851-862). The reasons for this dismal prognosis are its insidious onset (most patients present with nonspecific symptoms even in the presence of locally advanced, surgically unresectable disease or distant metastasis) and the modest efficacy of the adjuvant therapy. It has been estimated that earlier diagnosis of PDAC at a resectable stage could improve five-year survival up to almost 40% (Siegel R L, et al, CA Cancer J Clin. 2022 January; 72 (1): 7-33; Kenner B, et al, Pancreas. 2021 March; 50 (3): 251-279), emphasizing the importance of early diagnosis to reduce death rates. The only potentially curative treatment for PDAC is radical resection with tumour free margins although, because of the late diagnosis, less than 20% of the patients can benefit of this approach due to the presence of locally advanced disease or metastasis at the time of diagnosis (Gillen S, et al, PLOS Medicine. 2010 April; 7 (4): e1000267). Surveillance programs are recommended for groups at high-risk of developing PDAC: patients with germline mutations in DNA repair genes such BRCA1/2 or family history of pancreatic cancer (mostly PRSS1 mutations), patients with mucinous cysts (mucinous cystic neoplasms, MCN, and intraductal papillary mucinous neoplasms, IPMN), new onset-diabetes (patients diagnosed at age 50 and older) and patients with chronic pancreatitis. However, the screening of the general population for pancreatic cancer, as for other types of cancer, is not recommended due, among other factors, to the low sensitivity of the available tests for early stages.

Currently, the only clinically established biomarker for PDAC is Carbohydrate Antigen 19-9 (CA19-9). It is widely used in the monitoring of disease progression and the response to chemotherapy. However, it has a low positive predictive value due to the relatively low incidence of pancreatic cancer in asymptomatic subjects and lacks the specificity needed for early detection of the disease (Luo G, et al, Biochim Biophys Acta Rev Cancer. 2021 April; 1875 (2): 188409). Similar problems are found for early diagnosis of other cancer types such as gastric, esophageal, lung, liver or ovarian cancer.

Identification of biomarkers able to detect pancreatic cancer, such as PDAC, and other types of cancer at early stages using sensitive, specific, reliable, practical and cost-effective techniques is an urgent unmet medical need. Many studies have reported the use of tumor biopsy samples obtained during surgery or by ultrasound-guided fine needle aspiration or blood samples to identify genomic, proteomic, glycomic and metabolomics markers for cancer. However, none of these markers have been proved useful in the context of early diagnosis (surveillance biomarkers). On the other hand, tests described for early disease, such as a 29-protein marker signature (Mellby et al, J Clin Oncol. 2018 Oct. 1; 36 (28): 2887-2894), may have limited application to large populations because of their complexity.

There remains a need to provide predictive biomarker-based methods and tests for effectively diagnosing cancer which may be economically feasible for implementing in the clinical practice. Moreover, there is a need for these tools to be able to diagnose early-stage cancer.

### SUMMARY OF INVENTION

The present inventors have surprisingly found that a single metabolic signature based on the measurement of three metabolites may be used for diagnosing several types of cancer with good sensitivity and specificity.

Thus, a first aspect of the invention refers to an in vitro method for diagnosing cancer in a subject, said method comprising the steps of:
(i) determining, in a sample obtained from the subject, the concentration of (a) 3-oxopropanoate, and at least one further metabolite selected from (b) a ketone body selected from beta-hydroxybutyrate, acetoacetate, and combinations thereof, and (c) a medium chain fatty acid selected from octanoate, decanoate, and combinations thereof,
(ii) comparing the concentration of each of the metabolites determined in (i) with a corresponding reference value, and
(iii) diagnosing the patient as having cancer when the concentration of at least one of the metabolites is increased as compared to the corresponding reference value.

Generally, the reference value is, for each metabolite, the concentration of the metabolite determined in the biological sample obtained from a non-oncologic subject or group of patients.

The diagnosis requires determining (a) 3-oxopropanoate, and at least (b) beta-hydroxybutyrate or acetoacetate, or (c) octanoate or decanoate (herein referred to as MKM signature). However, it suffices that one of these metabolites is increased when compared to its corresponding reference value to provide a cancer diagnosis. This is because most of the patients suffering from cancer have increased blood concentrations of at least one of the metabolites. As shown in FIG. 7B, 87% blood samples of cancer patients (in this particular case, PDAC patients) included in the study had increased levels of at least one of the metabolites versus 6% in non-oncologic subjects. Many patients did not show increased levels of all metabolites but did have at least one of them. Looking for only one of the metabolites would only diagnose some cancer patients (FIG. 7D-F). However, by looking for the signature, most cancer patients are diagnosed because they either have one of the metabolites included in the signature or they have more than one, or they have them all. Altogether, looking for the combination of biomarkers as disclosed in the present invention significantly improved the discrimination of cancer patients versus non-oncologic patients.

Importantly, the present diagnosis method provides surprisingly good predictive values for cancer diagnosis. As shown in the examples below, the metabolic signature of the invention successfully discerned between non-oncologic controls (including healthy controls and chronic pancreatitis patients) and patients having pancreatic cancer, in this particular case, PDAC, with a ROC-AUC of 0.962 (95% CI 0.929-0.995) (FIG. 1). This result substantially improves the discriminating capabilities of CA 19.9 in the same samples (FIG. 2). The method of the invention outperforms prior diagnostic methods, in particular prior methods that make use of biomarkers.

Interestingly, the metabolic signature of the invention was able to discriminate between patients having chronic pancreatitis and patients with pancreatic cancer. As shown in FIG. 3B, a ROC-AUC of 0.862 (95% IC 0.743 to 0.986) was obtained when using MKM signature for comparison between early stage pancreatic cancer, in this particular case, PDAC, and chronic pancreatitis. This is an advantage for the clinical practice.

The metabolic signature of the invention also proved to distinguish non-oncologic patients from patients having other types of cancer with good sensitivities and specificities. For example, a ROC-AUC of 1.000 (95% IC 1.000-1.000) was obtained for gynecological cancer (FIG. 12) and a ROC-AUC of 0.996 (95% IC 0.989-1.000) was obtained for esophageal cancer (FIG. 10A). Lung cancer, both small cell lung cancer (SCLC) and non-small cell lung cancer (NSCLC) could also be diagnosed by using the metabolic signature of the invention (AUC: 0.797; 95% CI: 0.726-0.867; FIG. 13 for SCLC and AUC: 0.814, 95% IC: 0.731-0.894, FIG. 14 for NSCLC). Gastric cancer could also be diagnosed by using the metabolic signature of the invention (AUC: 0.760; 95% CI: 0.692-0.829; FIG. 8A). The signature was also able to diagnose cholangiocarcinomas (N=3, FIG. 16). The results demonstrate that the metabolic signature of the invention significantly improves the diagnosis of gastric, esophageal and gynecological cancer, SCLC, NSCLC, and cholangiocarcinoma.

Importantly, the present method can also detect cancer in early stages, which is of outmost importance for patient managing. This is particularly relevant for some cancers, such as pancreatic, gastric, esophageal, liver or ovarian cancer, which are most often detected in late, incurable, stages, while early diagnosis would often be the only hope of a successful therapeutic intervention. When comparing early stage pancreatic cancer, in this particular case, PDAC samples, with controls (healthy subjects and patients suffering from pancreatitis), the resulting ROC-AUC value was 0.963 (95% IC 0.923-1.000) (FIG. 3). This result substantially improves the discerning capabilities of CA 19.9 in the same samples (FIG. 4). FIGS. 9 and 11 further evidence the early-diagnosis potential of the present diagnosis method in gastric cancer and esophageal cancer.

The diagnosis method of the invention does not require providing a biopsied sample from the tumor. Instead, the diagnosis according to the invention may be performed, for example, by simply providing a blood sample, which is a far readily available biological sample. This method is also not invasive. Moreover, the present method is not limited to diagnosis of a particular type of cancer. The metabolic signature described herein has been found in different types of cancer. Consequently, several cancers may be diagnosed by the method of the invention.

Altogether, provided herein is a sensitive, specific, reliable, practical and cost-effective method for diagnosing cancer and, in particular, for diagnosing cancer in early stage. Moreover, the method of the invention may be used for cancer screening, including screening for early stage cancer.

A second aspect of the invention provides a kit of parts for diagnosing cancer as defined in the first aspect, said kit comprising: (i) means for determining the concentration of: (a) 3-oxopropanoate, and at least a further metabolite selected from (b) a ketone body selected from beta-hydroxybutyrate, acetoacetate and combinations thereof, and (c) a medium chain fatty acid selected from octanoate, decanoate and combinations thereof, and optionally means for determining the concentration of a further metabolite selected from (d) alpha-ketobutyrate, (e) gluconate, and combinations thereof,
(ii) reference values for the above metabolites, and
(iii) instructions for diagnosing cancer.

A third aspect refers to a combined use of (a) 3-oxopropanoate and at least one of (b) a ketone body selected from beta-hydroxybutyrate, acetoacetate and combinations thereof, or (c) a medium chain fatty acid selected from octanoate, decanoate and combinations thereof, as a biomarker for the diagnosis of cancer.

The invention further discloses a method for treating a subject suffering from cancer, said method comprising diagnosing cancer by an in vitro method as defined above and administering a medical regime for cancer to the patient if having cancer.

### BRIEF DESCRIPTION OF DRAWINGS

FIG. 1. ROC curve showing the potential of the diagnosis method of the invention (MKM signature containing the biomarkers 3-oxopropanoate, beta-hydroxybutyrate and decanoate) for the diagnosis of pancreatic cancer by using the whole PDAC cohort (patients diagnosed with both early and late stage PDAC).

FIG. 2. ROC curves showing the individual potential of (A) CA19.9, (B) decanoate Medium Chain Fatty Acid (MCFA), (C) 3-oxopropanoate and (D) beta-hydroxybutyrate (KB) for the diagnosis of pancreatic cancer by using the whole PDAC cohort (both early and late diagnosed patients).

FIG. 3. ROC curve showing the potential of the MKM signature containing the biomarkers 3-oxopropanoate, beta-hydroxybutyrate and decanoate for the early diagnosis of pancreatic cancer. (A) Healthy controls vs early stage (stages I and IIA) PDAC cohort. (B) Patients with chronic pancreatitis vs early stage (stages I and IIA) PDAC cohort

FIG. 4. ROC curves showing the individual potential of (A) CA19.9, (B) MCFA (decanoate), (C) 3-oxopropanoate and (D) KB (beta-hydroxybutyrate) for the early diagnosis of pancreatic cancer (stages I and IIA) by using the early stages PDAC cohort.

FIG. 5. ROC curve showing the potential of the 7-biomarker signature containing the biomarkers 3-oxopropanoate, beta-hydroxybutyrate, decanoate, octanoate, acetoacetate, gluconate and alpha-ketobutyrate for the diagnosis of pancreatic cancer by using the whole PDAC cohort (patients diagnosed with both early and late stage PDAC).

FIG. 6. ROC curve showing the potential of the 7-biomarker signature containing the biomarkers 3-oxopropanoate, beta-hydroxybutyrate, decanoate, octanoate, acetoacetate, gluconate and alpha-ketobutyrate for the early diagnosis of pancreatic cancer by using the early stage PDAC cohort. (A) Comparison between controls and early stages PDAC, (B) comparison between chronic pancreatitis and early stages PDAC.

FIG. 7. Percentage of PDAC patients having increased plasma concentration of at least one of the biomarkers of the 7-metabolite signature. (A) 7-metabolite signature in non-oncologic (healthy+pancreatitis) (N=36), (B) 7-metabolite signature in oncologic patients (PDAC) (N=80), (C) 7-metabolite signature+CA19.9 in oncologic (PDAC) patients (N=80). Percentage of PDAC patients having increased plasma concentrations of (D) 3-oxopropanoate, (E) one ketone body (beta-hydroxybutyrate or acetoacetate), (F) one medium chain fatty acid (octanoate or decanoate).

FIG. 8. ROC curve showing the diagnosis potential of the selected biomarkers in gastric cancer. (A) MKM signature containing the biomarkers 3-oxopropanoate, beta-hydroxybutyrate and decanoate for the diagnosis of gastric cancer by using the gastric cancer cohort, (B) 7-metabolite signature containing the biomarkers 3-oxopropanoate, beta-hydroxybutyrate, decanoate, octanoate, acetoacetate, gluconate and alpha-ketobutyrate for the diagnosis of gastric cancer by using the gastric cancer cohort.

FIG. 9. ROC curve showing the early-diagnosis potential of the selected biomarkers in gastric cancer. (A) MKM signature containing the biomarkers 3-oxopropanoate, beta-hydroxybutyrate and decanoate for the early-diagnosis of gastric cancer by using the gastric cancer cohort, (B) 7-metabolite signature containing the biomarkers 3-oxopropanoate, beta-hydroxybutyrate, decanoate, octanoate, acetoacetate, gluconate and alpha-ketobutyrate for the early-diagnosis of gastric cancer by using the gastric cancer cohort.

FIG. 10. ROC curve showing the diagnosis potential of the selected biomarkers in esophageal cancer. (A) MKM signature containing the biomarkers 3-oxopropanoate, beta-hydroxybutyrate and decanoate for the diagnosis of esophageal cancer by using the esophageal cancer cohort, (B) 7-metabolite signature containing the biomarkers 3-oxopropanoate, beta-hydroxybutyrate, decanoate, octanoate, acetoacetate, gluconate and alpha-ketobutyrate for the diagnosis of esophageal cancer by using the esophageal cancer cohort.

FIG. 11. ROC curve showing the early-diagnosis potential of the selected biomarkers in esophageal cancer. (A) MKM signature containing the biomarkers 3-oxopropanoate, beta-hydroxybutyrate and decanoate for the early-diagnosis of esophageal cancer by using the esophageal cancer cohort, (B) 7-metabolite signature containing the biomarkers 3-oxopropanoate, beta-hydroxybutyrate, decanoate, octanoate, acetoacetate, gluconate and alpha-ketobutyrate for the early-diagnosis of esophageal cancer by using the esophageal cancer cohort

FIG. 12. ROC curve showing the diagnosis potential of the selected biomarkers in gynecological cancer. (A) MKM signature containing the biomarkers 3-oxopropanoate, beta-hydroxybutyrate and decanoate for the diagnosis of gynecological cancer by using the gynecological cancer cohort, (B) 7-metabolite signature containing the biomarkers 3-oxopropanoate, beta-hydroxybutyrate, decanoate, octanoate, acetoacetate, gluconate and alpha-ketobutyrate for the diagnosis of gynecological cancer by using the gynecological cancer cohort.

FIG. 13. ROC curve showing the diagnosis potential of the selected biomarkers in small cell lung cancer (SCLC). (A) MKM signature containing the biomarkers 3-oxopropanoate, beta-hydroxybutyrate and decanoate for the diagnosis of SCLC by using the SCLC cohort, (B) 7-metabolite signature containing the biomarkers 3-oxopropanoate, beta-hydroxybutyrate, decanoate, octanoate, acetoacetate, gluconate and alpha-ketobutyrate for the diagnosis of SCLC by using the SCLC cohort.

FIG. 14. ROC curve showing the diagnosis potential of the selected biomarkers in non-small cell lung cancer (NSCLC). (A) MKM signature containing the biomarkers 3-oxopropanoate, beta-hydroxybutyrate and decanoate for the diagnosis of SCLC by using the NSCLC cohort, (B) 7-metabolite signature containing the biomarkers 3-oxopropanoate, beta-hydroxybutyrate, decanoate, octanoate, acetoacetate, gluconate and alpha-ketobutyrate for the diagnosis of SCLC by using the NSCLC cohort.

FIG. 15. Heatmaps showing in black the samples with an overproduced metabolite in an extended cohort of early stage pancreatic cancer patients, chronic pancreatitis patients, intraductal papillary mucinous neoplasm (IPMN) patients and control subjects.

FIG. 16. heatmap showing in black the samples with an overproduced metabolite in the Cholangiocarcinoma Cancer Cohort.

### DETAILED DESCRIPTION OF THE INVENTION

All terms as used herein in this application, unless otherwise stated, shall be understood in their ordinary meaning as known in the art. Other more specific definitions for certain terms as used in the present application are as set forth below and are intended to apply uniformly through-out the specification and claims unless an otherwise expressly set out definition provides a broader definition.

The term “diagnosis” is known to the person skilled in the art. As used herein “diagnosis” is understood as becoming aware of a particular medical condition, complication; the determination of the nature of the condition; or the distinguishing of the condition from another. It refers both to the process of attempting to determine or identify the possible condition, and to the opinion reached by this process. A diagnosis, in the sense of diagnostic procedure, can be regarded as an attempt at classification of an individual's condition into separate and distinct categories that allow medical decisions about treatment and prognosis to be made.

“Early-stage” when referring to cancer is generally understood as the first stages in cancer disease, in particular, it is referred to stages IA, IB, IIA and IIB, more particularly to stages IA, IB, IIA.

The term “biomarker”, as used herein, refers generally to a molecule, the concentration of which can be determined in a biological sample from a patient by standard methods (including those disclosed herein), and is predictive or denotes a condition of the patient from which it was obtained. In the present invention the biomarkers are one or more metabolites, or a combination of metabolites, the concentration of which may be determined, for example, by liquid chromatography coupled with tandem mass spectrometry or by enzymatic, colorimetric, fluorometric, luminometric, turbidimetric or other spectrophotometric method of analysis or diagnostic rapid strip test.

The term “reference value” in the context of the present invention is to be understood as a predefined concentration of the biomarker, in this case, the metabolite, in a sample or group of samples. The samples are taken from a non-oncologic subject or group of subjects, e.g., a healthy subject. This value is used as a threshold to discriminate subjects wherein the condition, cancer, to be analysed is present from those wherein such condition is absent. The skilled person in the art, making use of the general knowledge, is able to choose the subject or group of subjects more adequate for obtaining the reference value. Methods for obtaining the reference value from the group of subjects selected are well known in the state of the art (Burtis C. A. et al., 2008, Chapter 14, section “Statistical Treatment of Reference Values”). As will be apparent to the skilled person, the reference values will be defined according to the purpose, target population for the diagnosis, balance between specificity and sensitivity, etc. In some embodiments, the reference value may also be a particular threshold value above which it is considered that the patient has cancer with predetermined specificity and sensitivity.

In most embodiments of the invention the concentration of the metabolite is measured as the weight of metabolite per volume of sample. When the biological sample is blood, plasma or serum, which are convenient biological samples for the method of the invention, the concentration is usually measured in ng metabolite/mL biological sample.

In its more general concept, the invention refers to an in vitro method for diagnosing cancer in a subject which comprises: (i) determining, in a sample obtained from the subject, the concentration of (a) 3-oxopropanoate, and at least one further metabolite selected from (b) a ketone body, and (c) a medium chain fatty acid; (ii) comparing the concentration of each of the metabolites determined in (i) with a corresponding reference value; and (iii) diagnosing the patient as having cancer when the concentration of at least one of the metabolites is increased as compared to the corresponding reference value.

Ketone bodies are selected from beta-hydroxybutyrate (IUPAC name: 3-hydroxybutanoate), acetoacetate (IUPAC name 3-oxobutanoate), acetone (IUPAC name: propan-2-one), and combinations thereof. In some embodiments, the ketone body is selected from beta-hydroxybutyrate, acetoacetate, and combinations thereof. Medium chain fatty acids are preferably selected from octanoate, decanoate, and combinations thereof.

In a particular embodiment of the first aspect of the invention, step (i) comprises determining the concentration of (a) 3-oxopropanoate, (b) a ketone body selected from beta-hydroxybutyrate, acetoacetate and combinations thereof, and (c) a medium chain fatty acid selected from octanoate, decanoate, and combinations thereof.

In one embodiment, step (i) comprises determining the concentration of decanoate. In a particular embodiment, step (i) comprises determining the concentration of both decanoate and octanoate.

In one embodiment, step (i) comprises determining the concentration of beta-hydroxybutyrate. In a particular embodiment step (i) comprises determining the concentration of both beta-hydroxybutyrate and acetoacetate.

Thus, in a very particular embodiment of the invention, step (i) comprises determining 3-oxopropanoate, beta-hydroxybutyrate, acetoacetate, octanoate and decanoate.

As mentioned above, a significantly improved diagnosis as compared to prior methods that use biomarkers may be done by performing the method defined by the first aspect of the invention, which comprises determining: (a) 3-oxopropanoate, and (b) a ketone body selected from beta-hydroxybutyrate, acetoacetate and combinations thereof, or (c) a medium chain fatty acid selected from octanoate, decanoate and combinations thereof, in particular when determining (a), (b), and (c), more particularly when determining the concentration of 3-oxopropanoate, beta-hydroxybutyrate, acetoacetate, octanoate and decanoate. Nevertheless, the capacity to distinguish between non-oncologic and oncologic patients may be further improved by determining additional metabolites.

Appropriate metabolites for improving the method of the invention are alpha-ketobutyrate (IUPAC name: 2-oxobutanoate) and gluconate (IUPAC name: (2R,3S,4R,5R)-2,3,4,5,6-pentahydroxyhexanoate). Thus, in a more particular embodiment of the invention, step (i) further comprises determining the concentration of a metabolite selected from (d) alpha-ketobutyrate and (e) gluconate. In a still more particular embodiment, step (i) comprises determining 3-oxopropanoate, beta-hydroxybutyrate, acetoacetate, octanoate, decanoate, alpha-ketobutyrate, and gluconate.

In one embodiment, step (i) of the method further comprises determining the concentration of (d) alpha-ketobutyrate, (e) gluconate, or both. In a particular embodiment, the concentration of gluconate is determined. In a more preferred embodiment, the concentration of both gluconate and alpha-ketobutyrate is determined.

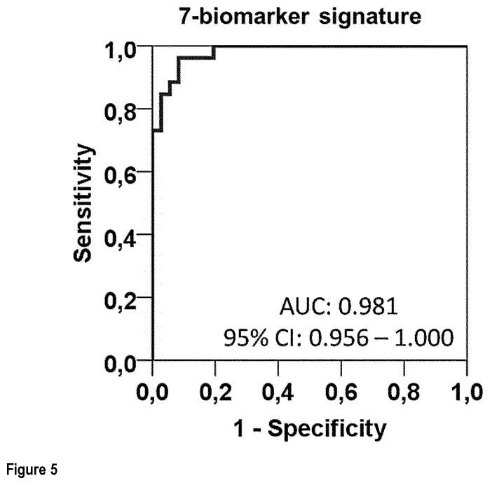

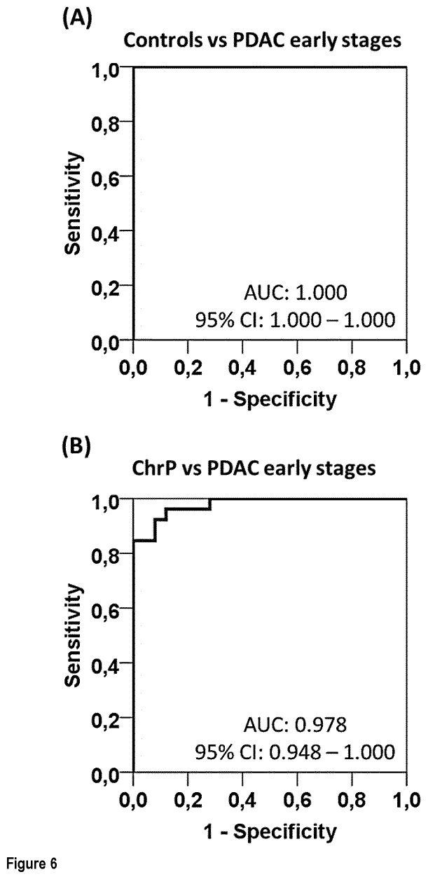

An “improved” diagnosis is understood as higher sensitivity and/or specificity which, as the skilled person would understand, may be determined by a higher ROC-AUC value. The inventors have found that the 7-metabolite signature is able to discriminate PDAC patients from controls with a receiver operating characteristic area under the curve (ROC-AUC) value of 0.981 (FIG. 5). The 7-metabolite signature was further able to distinguish early-stage PDAC patients from healthy controls with a ROC-AUC value of 1.000 ((95% IC 1.000 to 1.000; FIG. 6).

Additional biomarkers may be used to further enhance or complement the diagnosis of the invention. For example, the method may further comprise determining the concentration of another biomarker that has been disclosed for cancer diagnosis (or prognosis, or response to treatment) and comparing the determined concentration with a corresponding reference value. Thus, in one particular embodiment, step (i) of the method of the invention further comprises determining an additional marker related to cancer diagnosis or prognosis. Usually, in these cases, the patient may be diagnosed of having cancer with improved sensibility and/or specificity if said other biomarker is significantly increased or decreased when compared to its corresponding reference value. In a particular embodiment, the method of the invention further comprises determining CA19-9.

Appropriate additional biomarkers that can be used in the method of the invention may depend on the type of cancer. Non-limiting examples of biomarkers that may be useful for the method of the invention are provided below. For example, a biomarker selected from the group consisting of CA19-9, CA-242, CA-50, CA-195, CA 72-4, CEA and CA-125 may be used for diagnosing pancreatic cancer. In a particular embodiment, for diagnosing pancreatic cancer, the additional biomarker is CA19-9. A biomarker selected from the group consisting of Cytokeratin 19 fragment antigen 21-1 (Cyfra21-1), CA19-9, CA72-4, CEA and squamous cell carcinoma antigen (SCC-Ag) may be used for diagnosing esophageal cancer. In a particular embodiment, for diagnosing esophageal cancer, the additional biomarker is selected from CA19-9 and CEA. A biomarker selected from the group consisting of CA72-4, CA-50, alpha-fetoprotein, CA12-5, SLE, BCA-225, hCG and pepsinogen I/II may be used for diagnosing gastric cancer. In a particular embodiment, for diagnosing gastric cancer, the additional biomarker is selected from CEA and CA19-9. A biomarker selected from the group consisting of alpha-fetoprotein (AFP-L3), DCP/PVKA II, GPC3, GS, HSP70 (tissue), CK19, GP73, midkine, OPN, SCCA, Annexin A2 and FGF3/4 may be used for diagnosing liver cancer. In a particular embodiment, for diagnosing liver cancer, the additional biomarker is selected from alpha-fetoprotein (AFP-L3) and DCP/PVKA II. In another particular embodiment, for diagnosing cholangiocarcinoma, the additional biomarker is selected from CA19-9 and CA125. In another particular embodiment, for diagnosing ovarian cancer, the additional biomarker is CA125. In another particular embodiment, for diagnosing lung cancer, the additional biomarker is selected from NSE and CYFRA 21-1.

The sample obtained from the patient may be a tissue sample, or a biological sample (e.g., blood, plasma, serum, ascitic fluid, bronco-alveolar lavage, urine, breath, stool, CFS, saliva). In particular embodiments, the sample is a biological fluid. In more particular embodiments, the sample is selected from blood, plasma and serum. Early diagnosis is also facilitated by the invention, using devices such as the dry blood spots (DBS), which allow easy blood sample collection by simple pricking of a finger and mail transport to the reference center for its analysis without any special preservation conditions

The concentration of the metabolites may be determined by any analytical method available to the skilled person. In some embodiments, metabolites are determined using mass spectrometry. In some embodiments, metabolites are determined using liquid chromatography. In some embodiments, metabolites are determined using gas chromatography. In some embodiments, liquid chromatography in combination with mass spectrometry is used. In some embodiments, gas chromatography in combination with mass spectrometry is used. In particular embodiments, the concentration of the metabolites is determined by Liquid Chromatography with tandem mass spectrometry. The principle of tandem mass spectrometry is based on coupling mass spectrometers together in a series to analyze complex mixtures. The method uses two mass filters arranged sequentially with a collision cell between them. The filters can be used in static or scanning mode to select a particular mass-to-charge (m/z) ratio or m/z range. In the collision cell, the precursor ions collide with gas molecules and are fragmented into smaller ions referred to as product ions. Tandem mass spectrometers are versatile in that they can be operated using a variety of different scan modes depending on the clinical application. Exemplary mass spectrometry methodology is described in the examples.

In another embodiment, the concentration of the metabolites is determined by enzymatic analysis. Enzymatic determination of the herein disclosed metabolites is known in the art and can be performed by conventional and/or commercial means. In some embodiments, the enzymatic determination comprises oxidation of the metabolite in the presence of an appropriate enzyme and NAD⁺in combination with a detectable label.

For example, 3-oxopropanoate can be measured by end-point reaction catalyzed by Coenzyme A (CoA)-dependent 3-oxopropanoate dehydrogenases which transform 3-oxopropanoate into acetyl-CoA (for instance, human ALD6A1. The absorbance at 340 nm due to the formation of NADH is stochiometric with the amount of
3-oxopropanoate present in the sample:

3-oxopropanoate+NAD⁺+CoA→acetyl-CoA+NADH.

In another example, beta-hydroxybutyrate and acetoacetate can be measured by end-point reactions catalyzed by beta-hydroxybutyrate dehydrogenase:

beta-hydroxybutyrate+NAD⁺→acetoacetate+NADH

acetoacetate+NADH→beta-hydroxybutyrate+NAD⁺

The generation or consumption of NADH is measured spectrophotometrically at 340 nm and it is stochiometric with the amount of beta-hydroxybutyrate or acetoacetate, respectively, present in the sample.

In another example, acetoacetate can also be determined by the measurement of the formation of a colored complex with sodium nitroprusside at 550 nm.

In another embodiment gluconic acid can be measured by the coupled enzymatic reactions:
1. Phosphorylation of gluconic acid by gluconate kinase:

D-gluconate+ATP→D-gluconate-6-phosphate+ADP
2. Reduction of D-gluconate-6-phosphate by 6-phosphogluconate dehydrogenase:

D-gluconate-6-phosphate+NADP⁺→ribulose-5-phosphate+NADPH+CO₂+H⁺

The absorbance at 340 nm due to the formation of NADPH is stochiometric with the amount of gluconic acid present in the sample.

In another example, non-esterified fatty acids such as octanoic and decanoic acids can be measured by a coupled enzymatic assay based on the synthesis of their acyl-CoA derivatives by Acyl-CoA synthetases and their posterior oxidation by Acyl-CoA dehydrogenases with colorimetric quantification of the H₂O₂ produced. In a more detailed procedure:
(1) Acyl-CoA synthesis: fatty acid activation using an Acyl-CoA synthetase with narrow substrate specificity. For example, an Acyl-CoA synthetase active with fatty acids C5 to C14 but poorly active with longer chain fatty acids:

R—COOH+ATP+CoA→R—CO-S-CoA+AMP+PPi
(2) Oxidation of the acyl-CoA by medium-chain acyl-CoA dehydrogenases. For example, hACADM, which acts specifically on acyl-CoAs with saturated 6 to 12 carbons-long primary chains:

R-CH₂-CH₂—CO—S-CoA+O2+FAD→R—CH═CH—CO—S-CoA+H₂O₂+FADH
(3) Quantification of the H₂O₂ produced, for example:

H₂O₂+4-amino antipyrine+2,4-dibromophenol→quinoneimine dye, catalyzed by horseradish POD.

In yet another example, alpha-ketobutyrate can be quantified by monitoring the formation of 2-ethyl-2-quinoxalinol upon reaction with o-phenylendiamine by measuring the absorbance at 334 nm (Li R and Kenyon G L. Anal Biochem. 1995 23037-40).

Step (ii) of the diagnosis method of the invention comprises comparing the concentration of each of the metabolites determined in (i) with a corresponding reference value. The reference value for each metabolite is the concentration of said metabolite determined in the biological sample obtained from a non-oncologic subject or group of subjects (controls). In some embodiments, the reference value for each metabolite is the concentration of said metabolite determined in the biological sample obtained from a healthy subject or group of subjects. The reference value for each metabolite may be determined as described above. The inventors have determined exemplary reference values for each biomarker by determining the concentration in a control group of non-oncologic subjects. Based on these findings, reference values (reference concentrations) may be established. In one embodiment, the reference value for 3-oxopropanoate is in the range from 15 to 30 ng/ml, more particularly from 15 to 25 ng/ml. In one embodiment, the reference value for beta-hydroxybutyrate is in the range from 6000 to 10000 ng/ml, more particularly from 7000 to 9000 ng/ml. In one embodiment, the reference value for acetoacetate is in the range from 1000 to 3000 ng/ml, more particularly from 2000 to 3000 ng/ml. In one embodiment, the reference value for octanoate is in the range from 50 to 200 ng/ml, more particularly from 90 to 150 ng/ml. In one embodiment, the reference value for decanoate is in the range from 100 to 400 ng/ml, more particularly from 200 to 350 ng/ml. In one embodiment, the reference value for alpha-ketobutyrate is in the range from 120 to 200 ng/ml, in particular from 140 to 190 ng/ml. In a particular embodiment, the reference value for gluconate is in the range from 800 to 1500 ng/ml, more particularly from 900 to 1300 ng/ml. The above values may provide a clear information for performing the comparing step (ii) as defined in the method of the invention. However, the skilled person may use any available method to establish said comparison.

Step (iii) of the diagnosis method of the invention comprises diagnosing the patient as having cancer when the concentration of at least one of the metabolites is increased as compared to the corresponding reference value.

In one embodiment, the patient is diagnosed with cancer if at least one of the metabolites is increased at least 2-fold when compared to the corresponding reference value. In a particular embodiment, the increase is at least 3-fold. In another particular embodiment, the increase is at least 4-fold. In another particular embodiment, the increase is at least 5-fold. In another particular embodiment, the increase is at least 6-fold. In another particular embodiment, the increase is at least 10-fold. In a more particular embodiment, the increase is at least 10-fold. In an even more particular embodiment, the increase is at least 20-fold, for example at least 30-fold or at least 50-fold.

In one embodiment, the patient is diagnosed with cancer if at least one of the metabolites is increased at least 1.5%, at least 2%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%: at least 85%, at least 90%, at least 95%, at least 100%, at least 110%, at least 120%, at least 130%, at least 140%, at least 150%, at least 200%, at least 300%, at least 400%, at least 500%, or at least 600% or more higher than the reference value.

Based on the reference values, threshold values may be provided for each of the metabolites. The “threshold value”, also referred to as “cut-off value”, for a metabolite is understood as a concentration value above which the patient is diagnosed as having cancer (according to the method of the invention) with good sensitivity and specificity. Thus, in some embodiments, the reference value is a threshold value. In these embodiments, step (ii) of the method of the invention comprises comparing the concentration of each of the metabolites determined in (i) with a corresponding threshold value and step (iii) comprises diagnosing the patient as having cancer when the concentration of at least one of the metabolites is increased as compared to the corresponding threshold value.

In one embodiment, the threshold value for 3-oxopropanoate is from 50 ng/ml to 500 ng/ml, in particular from 70 to 300 ng/ml, more particularly from 150 to 300 ng/ml, and even more particularly, around 250 ng/ml. In one embodiment, the threshold value for beta-hydroxybutyrate is from 25000 to 70000 ng/ml, in particular from 30000 to 60000 ng/ml, more particularly from 40000 to 55000 ng/ml, and even more particularly, around 50000 ng/ml. In one embodiment, the threshold value for acetoacetate is from 9000 to 15000 ng/ml, in particular from 10000 to 13500 ng/ml, more particularly from 11500 to 13000 ng/ml, and even more particularly, around 12500 ng/ml. In one embodiment, the threshold value for octanoate is from 750 to 4000 ng/mL, in particular from 1000 to 3000 ng/ml, more particularly from 1500 to 2500 ng/ml, and even more particularly, around 2000 ng/ml. In one embodiment, the threshold value for decanoate is from 1300 to 4500 ng/ml, in particular from 1500 to 4000 ng/ml, more particularly from 2000 to 3500 ng/ml, and even more particularly, around 3000 ng/ml. In one embodiment, the threshold value for alpha-ketobutyrate is from 400 to 1000 ng/ml, in particular from 500 to 900 ng/ml, more particularly from 600 to 800 ng/ml, and even more particularly, around 700 ng/ml. In one embodiment, the threshold value for gluconate is from 3000 to 6500 ng/ml, in particular from 3500 to 6000 ng/ml, more particularly from 4000 to 5500 ng/mL, and even more particularly, around 5000 ng/ml. In one embodiment, the threshold value for octanoate+decanoate is from 2000 to 5000 ng/ml, in particular from 3000 to 4500 ng/ml, more particularly around 3500 ng/ml, and even more particularly, around 4000 ng/ml. In one embodiment, the threshold value for beta-hydroxybutyrate+acetoacetate is from 40000 to 60000 ng/ml, in particular around 45000 ng/ml, more particularly around 50000 ng/mL, and even more particularly, around 55000 ng/ml.

The method of the invention requires that at least one the metabolites determined in step (i) is increased in order to diagnose cancer. However, it is of course possible that two, three, four, five, six or seven metabolites are increased when compared with their corresponding reference values. In a particular embodiment, at least (a), i.e., at least 3-oxopropanoate, is increased as compared to the corresponding reference value. In a more particular embodiment at least (a) and (b) are increased. In another more particular embodiment at least (a) and (c) are increased. In an even more particular embodiment, (a), (b) and (c) are increased. In a still more particular embodiment, 3-oxopropanoate, beta-hydroxybutyrate, acetoacetate, octanoate, decanoate, and, optionally, alpha-ketobutyrate and gluconate are increased. The metabolite (a) and at least one of (b) or (c) are determined. However, as explained above, it is enough that only one of (a), (b), or (c) is higher than a corresponding reference value to differentiate between oncologic and non-oncologic patients.

As outlined above, the method of the invention outperforms prior diagnostic methods, in particular prior methods that make use of biomarkers. The performance of the diagnosis method, in this case, the ability to discriminate between oncologic and non-oncologic patients, may be defined by its sensitivity and specificity, which may be in turn determined by a receiver operating characteristic (ROC) curve and by calculating the corresponding area under the curve (AUC). In one embodiment, the method for diagnosing cancer of the invention is characterized by a AUC-ROC value in the range from 0.750 to 1.000. In a particular embodiment, the method is characterized by a AUC-ROC value in the range above 0.800. In a more particular embodiment, the method is characterized by a AUC-ROC value in the range above 0.850. In an even more particular embodiment, the method is characterized by a AUC-ROC value above 0.900. In an even more particular embodiment, the method is characterized by a AUC-ROC value above 0.950, more particularly above 0.960, more particularly above 0.970, more particularly above 0.980.

The above AUC-ROC values determine the sensitivity and specificity of the method. In a particular embodiment, the sensitivity of the method of the invention ranges from 75.00% to 100.00% or from 75.00% to 99.00%. In a more particular embodiment, the sensitivity of the method of the invention is above 80.00%. In an even more particular embodiment, the sensitivity of the method of the invention is above 85%. In an even more particular embodiment the sensitivity is above 87%, for example 88%, 89%, 90%, 91%, 92%, 93%, 94%, 95%, 96%, 97%, 98% or 99%. In a particular embodiment, the specificity of the method of the invention ranges from 75.00% to 100.00% or from 75.00% to 99.00%. In a more particular embodiment, the specificity of the method of the invention is above 80.00%. In an even more particular embodiment, the specificity of the method of the invention is above 85%. In an even more particular embodiment the specificity is above 87%, for example 88%, 89%, 90%, 91%, 92%, 93%, 94%, 95%, 96%, 97%, 98% or 99%.

As outlined above, the method of the invention is particularly suited for diagnosing cancer in early stages. It cannot be sufficiently stressed how important it is to have tools for diagnosing early stage cancer. It is well known that early diagnosis increases the chances of therapeutic success enormously. In some cases, such as pancreatic cancer, early diagnosis is often the only hope of a successful therapeutic intervention. Thus, in a particular embodiment, the method of the invention is for diagnosing early-stage cancer, in particular, cancer in stages IA, IB and IIA. All the embodiments disclosed above for cancer diagnosis (in general), including AUC-ROC values, sensitivity and specificity values, also apply to the method of the invention when diagnosing early-stage cancer.

The method of the invention may be used to diagnose most types of cancers. In one embodiment, the method of the invention is for diagnosing solid cancers. Solid cancers are defined as abnormal masses of tissue that usually do not contain cysts or liquid areas. They grow in organ systems such as pancreas, stomach, liver, lung, esophagus, breast or ovaries and can occur anywhere in the body. Examples of solid tumors are sarcomas, carcinomas, and lymphomas. In particular embodiments, the method of the invention is for diagnosing a cancer selected from the group consisting of pancreatic cancer, gastric cancer, esophageal cancer, liver cancer, gynecological cancer, breast cancer, colorectal cancer, and lung cancer. In more particular embodiments, the method of the invention is for diagnosing pancreatic cancer, gastric cancer, esophageal cancer, lung cancer, liver cancer or gynecological cancer. In an even more particular embodiment, the method of the invention is for diagnosing pancreatic cancer, for example pancreatic ductal adenocarcinoma. It is a very significant advantage if the method of the invention that cancer, including pancreatic cancer, gastric cancer or esophageal cancer, can be diagnosed in an early stage.

Further provided is a method for treating a subject suffering from cancer, said method comprising diagnosing cancer by an in vitro method as defined above and administering an appropriate therapeutic regime for cancer to the patient if diagnosed with cancer. For example, the therapeutic regime may be surgical resection, radiotherapy, chemotherapy, immunotherapy, hormonal therapy, hyperthermia, photodynamic therapy, stem cell transplant or combinations thereof. Thus, in particular embodiments the method is for diagnosing early-stage cancer, so it provides useful information for the clinician to choose the therapeutic regime to be administered or indicates the one that is best suited or only possible for patients having cancer in early stages. In particular, the only curative therapeutic regime may be surgical resection in the case of pancreatic, gastric, esophageal, liver and lung cancer if they are diagnosed at an early stage. This is especially relevant in many cases for which no other effective therapy is available, such as pancreatic, gastric, esophageal, lung or liver cancer. For example, when the diagnosis is of early-stage PDAC, the patient may be treated with curative intention by radical resection with tumor free margins.

Also disclosed is a kit of parts comprising means for determining the concentration of (a) 3-oxopropanoate, and another metabolite selected from (b) a ketone body selected from beta-hydroxybutyrate, acetoacetate and combinations thereof and (c) a medium chain fatty acid selected from octanoate, decanoate and combinations thereof, and optionally means for determining the concentration of a further metabolite selected from (d) alpha-ketobutyrate, (e) gluconate, and combinations thereof. Said means may include reagents and buffers for sample treatment, reagents and buffers for metabolite extraction and derivatization, standards for calibration, elution buffers, chromatography columns, enzyme solutions, substrates, colorimetric, luminometric, turbidimetric or fluorometric labels, solid supports, etc. The kit may also comprise reference values for the above metabolites, and it often comprises instructions enabling the comparison of the determined biomarkers with reference the values in order to make the diagnosis. In most cases the kit comprises information about threshold values, calibration, sample treatment, assay conditions, etc. The kit may also contain a blood extraction device, for example, a dry blood spots (DBS), which allows easy blood sample collection by simple pricking of a finger alone or in combination with diagnostic rapid strip tests.

In one embodiment, the kit comprises instructions to determine if the patient has cancer. Said instructions typically comprise an explanation about how to perform the determination and how to provide a diagnosis according to the present method. This could also comprise, for example, reference concentrations for each metabolite to be determined and instructions as to how to compare the determined metabolite concentration in the patient's sample with the reference. It may also comprise cut-off values above which the determined concentration of metabolites is considered to indicate that the patient has cancer.

The present invention further provides for the use of the kits as defined above for the diagnosis of cancer according to the method of the invention.

The method of the invention may be automated in order to provide a diagnosis result. Thus, the invention also provides a system for determining the diagnosis of cancer in a patient comprising data processing means, said data processing means been configured:
to assess in a test sample obtained from the patient the concentration of (a) and either (b) or (c) or both (b) and (c), and optionally a further metabolite selected from (d), (e) or both (d) and (e);
to determine whether said concentrations are significantly up-regulated as compared corresponding reference values; and
to determine the diagnosis of cancer by evaluating the result of the previous determination.

The in vitro method of the invention generally provide for determining the diagnosis of cancer in a patient and for recommending an appropriate medical regime for the treatment of said patient. In some embodiments, said method may further comprise the steps of (i) collecting the diagnostic information, and (ii) saving the information in a data carrier.

In the sense of the invention a “data carrier” is to be understood as any means that contain meaningful information data for determining the diagnosis of cancer, in particular, of early-stage cancer, in a patient and/or recommending an appropriate medical regime, such as paper. The carrier may also be any entity or device capable of carrying the prognosis data or information for recommending an appropriate therapy. For example, the carrier may comprise a storage medium, such as a ROM, for example a CD ROM or a semiconductor ROM, or a magnetic recording medium, for example a floppy disc or hard disk. Further, the carrier may be a transmissible carrier such as an electrical or optical signal, which may be conveyed via electrical or optical cable or by radio or other means. When the diagnosis data are embodied in a signal that may be conveyed directly by a cable or other device or means, the carrier may be constituted by such cable or other device or means. Other carriers relate to USB devices and computer archives. Examples of suitable data carrier are paper, CDs, USB, computer archives in PCs, or sound registration with the same information. For completeness, the present description is also disclosed in the following numbered embodiments.

1. An in vitro method for diagnosing cancer in a subject, said method comprising:
(i) determining, in a sample obtained from the subject, the concentration of (a) 3-oxopropanoate, and at least one further metabolite selected from (b) a ketone body, and (c) a medium chain fatty acid;
(ii) comparing the concentration of each of the metabolites determined in (i) with a corresponding reference value; and
(iii) diagnosing the patient as having cancer when the concentration of at least one of the metabolites is increased as compared to the corresponding reference value.

2. The in vitro method for diagnosing cancer in a subject according to the preceding embodiment, said method comprising the steps of:
(i) determining, in a sample obtained from the subject, the concentration of (a) 3-oxopropanoate, and at least one further metabolite selected from (b) a ketone body selected from beta-hydroxybutyrate, acetoacetate, and combinations thereof, and (c) a medium chain fatty acid selected from octanoate, decanoate, and combinations thereof;
(ii) comparing the concentration of each of the metabolites determined in (i) with a corresponding reference value; and
(iii) diagnosing the patient as having cancer when the concentration of at least one of the metabolites is increased as compared to the corresponding reference value.

3. The in vitro method according to any one of the preceding embodiments, wherein step (i) comprises determining (a), (b) and (c).

4. The in vitro method according to any one of the preceding embodiments, wherein step (i) comprises determining the concentration of decanoate.

5. The in vitro method according to any one of the preceding embodiments, wherein step (i) comprises determining the concentration of both decanoate and octanoate.

6. The in vitro method according to any one of the preceding embodiments, wherein step (i) comprises determining the concentration of beta-hydroxybutyrate.

7. The in vitro method according to any one of the preceding embodiments, wherein step (i) comprises determining the concentration of both beta-hydroxybutyrate and acetoacetate.

8. The in vitro method according to any one of the preceding embodiments, wherein step (i) comprises determining 3-oxopropanoate, beta-hydroxybutyrate, acetoacetate, octanoate and decanoate.

9. The in vitro method according to any one of the preceding embodiments, wherein at least one metabolity is increased as compared to the corresponding reference value, in particular, said metabolite is 3-oxopropanoate.

10. The in vitro method according to any one of the preceding embodiments, wherein at least two metabolites are increased as compared to the corresponding reference values.

11. The in vitro method according to any one of the preceding embodiments, wherein at least three, four or five metabolites are increased as compared to the corresponding reference values.

12. The in vitro method according to any one of the preceding embodiments, wherein step (i) further comprises determining the concentration of (d) alpha-ketobutyrate.

13. The in vitro method according to any one of the preceding embodiments, wherein step (i) further comprises determining the concentration of (e) gluconate.

14. The in vitro method according to any one of the preceding embodiments, wherein step (i) comprises determining 3-oxopropanoate, beta-hydroxybutyrate, acetoacetate, octanoate, decanoate, alpha-ketobutyrate, and gluconate.

15. The in vitro method according to any one of the preceding embodiments, wherein the reference value for each metabolite is the concentration of said metabolite determined in the biological sample obtained from a non-oncologic subject or group of subjects.

16. The in vitro method according to any one of the preceding embodiments, wherein the increase is at least 3-fold, in particular at least 4-fold, more in particular at least 5-fold.

17. The in vitro method according to the preceding embodiment, wherein the increase is at least 10-fold, in particular at least 20-fold, more in particular at least 30-fold, even more in particular at least 50-fold.

18. The in vitro method according to any one of embodiments 1-15, wherein the reference value is a threshold value.

19. The in vitro method according to the preceding embodiment, wherein the threshold value for 3-oxopropanoate is from 150 to 300 ng/ml, in particular, around 250 ng/mL.

20. The in vitro method according to any one of embodiments 18-19, wherein the threshold value for beta-hydroxybutyrate is from 40000 to 55000 ng/ml, in particular, around 50000 ng/ml.

21. The in vitro method according to any one of embodiments 18-20, wherein the threshold value for acetoacetate is from 11500 to 13000 ng/mL, in particular, around 12500 ng/ml.

22. The in vitro method according to any one of embodiments 18-21, wherein the threshold value for octanoate is from 1500 to 2500 ng/ml, in particular, around 2000 ng/ml.

23. The in vitro method according to any one of embodiments 18-22, wherein the threshold value for decanoate is from 2000 to 3500 ng/ml, in particular, around 3000 ng/ml.

24. The in vitro method according to any one of embodiments 18-23, wherein the threshold value for alpha-ketobutyrate is from 600 to 800 ng/ml, in particular, around 700 ng/ml.

25. The in vitro method according to any one of embodiments 18-24, wherein the threshold value for gluconate is from 4000 to 5500 ng/ml, in particular, around 5000 ng/ml.

26. The in vitro method according to any one of embodiments 18-25, wherein the threshold value for octanoate+decanoate is from 3000 to 4500 ng/ml, in particular, around 4000 ng/ml.

27. The in vitro method according to any one of embodiments 18-26, wherein the threshold value for beta-hydroxybutyrate+acetoacetate is from 40000 to 60000 ng/ml, in particular, around 55000 ng/ml.

28. The in vitro method according to any one of the preceding embodiments, wherein the method is characterized by a receiver operating characteristic area under the curve value in the range from 0.700 to 1.000, in particular in the range from 0.800 to 1.000.

29. The in vitro method according to the preceding embodiment, wherein the method is characterized by a receiver operating characteristic area under the curve value in the range from 0.900 to 1.000, in particular in the range from 0.950 to 1.000.

30. The in vitro method according to any one of the preceding embodiments, wherein the sensitivity of the method of the invention is above 70.00%.

31. The in vitro method according to the preceding embodiment, wherein the sensitivity of the method of the invention is above 80.00%.

32. The in vitro method according to the preceding embodiment, wherein the sensitivity of the method of the invention is above 90.00%.

33. The in vitro method according to the preceding embodiment, wherein the sensitivity of the method of the invention is above 95.00%.

34. The in vitro method according to the preceding embodiment, wherein the sensitivity of the method of the invention is above 98.00%.

35. The in vitro method according to the preceding embodiment, wherein the specificity of the method of the invention is above 70.00%.

36. The in vitro method according to the preceding embodiment, wherein the specificity of the method of the invention is above 80.00%.

37. The in vitro method according to the preceding embodiment, wherein the s specificity of the method of the invention is above 90.00%.

38. The in vitro method according to the preceding embodiment, wherein the specificity of the method of the invention is above 95.00%.

39. The in vitro method according to any the preceding embodiment, wherein the specificity of the method of the invention is above 98.00%.

40. The in vitro method according to any one of the preceding embodiments, wherein the subject is diagnosed as having early stage cancer, in particular, cancer in stages IA, IB or IIA.

41. The in vitro diagnosis according to any one of the preceding embodiments, that is for diagnosing a solid tumor.

42. The in vitro diagnosis according to the preceding embodiment, wherein the cancer is selected from the group consisting of sarcomas, carcinomas, and lymphomas.

43. The in vitro diagnosis according to the preceding embodiment, wherein the cancer is selected from the group consisting of pancreatic cancer, gastric cancer, esophageal cancer, gynecological cancer, liver cancer, breast cancer, colorectal cancer, and lung cancer, in particular pancreatic cancer, more particularly pancreatic ductal adenocarcinoma.

44. The in vitro diagnosis according to any one of the preceding claims, wherein the sample is selected from blood, plasma, serum, ascitic fluid, bronco-alveolar lavage, urine, breath, stool, CFS, and saliva

45. The in vitro diagnosis according to the preceding embodiment, wherein the sample is selected from blood, plasma and serum.

46. The in vitro diagnosis according to any one of the preceding embodiments, wherein the amount of metabolite is determined by Liquid Chromatography with tandem mass spectrometry, colorimetry, fluorimetry, luminometry, turbidimetry or other type of spectrophotometric technique enzymatic or non-enzymatic including diagnostic rapid strip tests.

47. A kit of parts comprising:
(i) means for determining the concentration of (a) 3-oxopropanoate, and at least one other metabolite selected from (b) a ketone body selected from beta-hydroxybutyrate, acetoacetate and combinations thereof, and (c) a medium chain fatty acid selected from octanoate, decanoate and combinations thereof, and optionally means for determining the concentration of (d) alpha-ketobutyrate, (e) gluconate, and combinations thereof,
(ii) optionally, reference values for the above metabolites, and
(iii) instructions for diagnosing cancer.

48. The kit of parts according to the preceding embodiment, that comprises means for determining the concentration of 3-oxopropanoate, beta-hydroxybutyrate, acetoacetate, octanoate, and decanoate.

49. The kit of parts according to the preceding embodiment, additionally comprising means for determining the concentration of alpha-ketobutyrate and gluconate.

50. A kit of parts as defined in any one of embodiments 47-49 for diagnosing cancer as defined in any one of embodiments 1-46.

51. Combined use of (a) 3-oxopropanoate and at least one of (b) a ketone body selected from beta-hydroxybutyrate, acetoacetate and combinations thereof, or (c) a medium chain fatty acid selected from octanoate, decanoate and combinations thereof, as a biomarker for the diagnosis of cancer.

52. Combined use of 3-oxopropanoate, beta-hydroxybutyrate, acetoacetate, octanoate, and decanoate, as a biomarker for the diagnosis of cancer.

53. Combined use of 3-oxopropanoate, beta-hydroxybutyrate, acetoacetate, octanoate, decanoate, alpha-ketobutyrate and gluconate as a biomarker for the diagnosis of cancer.

Throughout the description and claims the word “comprise” and variations of the word, are not intended to exclude other technical features, additives, components, or steps. Furthermore, the word “comprise” encompasses the case of “consisting of”. Additional objects, advantages and features of the invention will become apparent to those skilled in the art upon examination of the description or may be learned by practice of the invention. The following examples are provided by way of illustration, and they are not intended to be limiting of the present invention. Furthermore, the present invention covers all possible combinations of particular and preferred embodiments described herein.

### Examples

### 1. Analysis of the Metabolic Signature in Cancer Patients by Liquid Chromatography-Tandem Mass Spectrometry (LC-MS/MS)

### 1.1. Methods

### Patients

Pancreatic cancer. The Pancreatic Cancer Cohort comprised 80 PDAC patients, 25 chronic pancreatitis (ChrP) patients and 11 healthy controls (HC), was collected at Hospital Universitari Doctor Josep Trueta (Girona, Spain). After diagnosis samples were classified as shown in Table 1. The overall resection rate was 89%. Serum samples were collected the day before surgery for patients with Stages I/II and the day before starting chemotherapy for patients with Stages III/IV PDAC.

TABLE 1
Patient demographics of the Pancreatic Cancer.

| | |
|---|---|
| PDAC | Pancreatic Cancer Cohort |

| | | |
|---|---|---|
| AJCC | Sex | Median age |

| | STAGE | n= | Male | Female | years (range) |
|---|---|---|---|---|---|
| | I | 9 | 5 | 4 | 73 (56-80) |
| | IIA | 17 | 9 | 8 | 70 (55-79) |
| | IIB | 25 | 7 | 18 | 67 (49-81) |
| | III | 13 | 10 | 3 | 64 (48-73) |
| | IV | 16 | 9 | 7 | 67 (30-81) |
| ChrP | | 25 | 19 | 6 | 55 (39-75) |
| HC | | 11 | 6 | 5 | 62 (44-75) |

Gastric cancer. The Gastric Cancer Cohort comprised sera from 127 patients diagnosed with adenocarcinomas from the stomach or the gastroesophageal junction (Siewert type III) who underwent surgery during the period 2004-2016 at the Gastro-Intestinal Surgery Department from Hospital del Mar, (Barcelona, Spain) and 52 healthy controls (HC) collected the Hospital del Mar (n=41) and Hospital Universitari Doctor Josep Trueta (n=11, Girona, Spain). After diagnosis samples were classified as shown in Table 2. Serum samples were collected the day before starting neoadjuvant treatment or before surgery in the case of the patients who did not receive it.

TABLE 2
Patient demographics of the Gastric Cancer Cohort

| | |
|---|---|
| GC | Gastric Cancer Cohort |

| | | |
|---|---|---|
| AJCC | Sex | Median age |

| | STAGE | n= | Male | Female | years (range) |
|---|---|---|---|---|---|
| | 0 | 1 | 1 | | 73 |
| | IA | 15 | 10 | 5 | 73 (69-86) |
| | IB | 16 | 8 | 8 | 78 (49-89) |
| | IIA | 16 | 8 | 8 | 78 (32-89) |
| | IIB | 15 | 9 | 6 | 73 (38-86) |
| | IIIA | 12 | 6 | 6 | 75 (59-84) |
| | IIIB | 19 | 12 | 7 | 73 (52-90) |
| | IIIC | 20 | 14 | 6 | 70 (42-90) |
| | IV | 13 | 6 | 7 | 74 (50-88) |
| HC | | 52 | 39 | 13 | 63 (27-81) |

Esophageal cancer. The Esophageal Cancer Cohort comprised sera from 52 patients diagnosed with adenocarcinomas or squamous cell carcinomas from the esophagus or the gastroesophageal junction (Siewert types I and II) who underwent surgery during the period 2006-2019 at the gastro-Intestinal Surgery Department from Hospital del Mar, (Barcelona, Spain) and 50 healthy controls (HC) collected at the Blood Bank from Hospital Vall d′Hebron (Barcelona). Serum samples were collected the day before starting the neoadjuvant treatment.

TABLE 3
Patient demographics of the Esophageal Cancer Cohort

| | | |
|---|---|---|
| ES | Esophageal Cancer Cohort | years |

| | | |
|---|---|---|
| AJCC | Sex | mean |

| | STAGE | n= | male | female | (range) |
|---|---|---|---|---|---|
| | 0 | 7 | 5 | 2 | 59 (42-77) |
| | I | 9 | 8 | 1 | 58 (41-79) |
| | II | 8 | 6 | 2 | 71 (57-80) |
| | III | 19 | 16 | 3 | 68 (40-83) |
| | IV | 4 | 4 | 0 | 65 (64-68) |
| | NA | 5 | NA | NA | NA |
| HC | — | 50 | 33 | 17 | 62 (51-69) |

Small Cell Lung Cancer Cohort. The Small Cell Lung Cancer (SCLC) Cohort comprised sera from 98 patients diagnosed with small cell lung cancer at the Medical Oncology Department at Hospital del Mar (Barcelona, Spain) and 50 healthy controls (HC) collected at the Blood Bank from Hospital Vall d′Hebrón (Barcelona).

TABLE 4
Patient demographics of the SCLC Cancer Cohort

| | |
|---|---|
| EC | SCLC Cohort |

| | | |
|---|---|---|
| AJCC | Sex | Median age |

| | STAGE | n= | Male | Female | years (range) |
|---|---|---|---|---|---|
| | I | 3 | 1 | 2 | 76 (76-81) |
| | III | 3 | 3 | 0 | 77 (57-78) |
| | III | 30 | 17 | 13 | 67 (45-87) |
| | IV | 62 | 48 | 14 | 68 (51-88) |
| HC | | 50 | 33 | 17 | 61 (51-69) |

Non-Small Cell Lung Cancer Cohort. The Non-Small Cell Lung Cancer (NSCLC) Cohort comprised sera from 92 patients diagnosed with non-small cell lung cancer at the Medical Oncology Department at Hospital del Mar (Barcelona, Spain) and 50 healthy controls (HC), 22 collected at Hospital del Mar and 28 at the Blood Bank from Hospital Vall d′Hebrón (Barcelona).

TABLE 5
Patient Demographics of the NSCLC Cohort

| | |
|---|---|
| EC | NSCLC Cohort |

| | | |
|---|---|---|
| AJCC | Sex | Median age |

| | STAGE | n= | Male | Female | years (range) |
|---|---|---|---|---|---|
| | I | 4 | 3 | 1 | 69 (59-72) |
| | III | 10 | 8 | 2 | 70 (58-82) |
| | III | 22 | 19 | 3 | 65 (53-81) |
| | IV | 56 | 47 | 9 | 66 (44-83) |
| HC | | 50 | UNK | UNK | 59 (29-81) |

Gynecological cancer. The Gynecological Cancer Cohort comprised sera from 10 patients diagnosed with endometrial (N=4), ovarian (N=2), cervical (N=2) and uterine (N=1) cancer and 6 HC sera collected at Hospital del Mar.

TABLE 6
Patient demographics of the Gynecological Cancer Cohort.

Gynecological Cancer Cohort

| | cancer | Stage | | | Median age |
|---|---|---|---|---|---|
| | type | I | II | III | years (range) |
| | endometrial | 4 | — | 1 | 59 (43-73) |
| | ovarian | — | — | 2 | 68 (67-68) |
| | cervical | 1 | 1 | — | 51 (46-57) |
| | uterine | 1 | — | — | 60 |
| HC | n = 6 | NA | NA | NA | 56 (45-79) |

### Cholangiocarcinoma.

TABLE 7
Patient Demographics of the Cholangiocarcinoma Cancer Cohort

| | |
|---|---|
| Cholangiocarcinoma Cancer Cohort | years |

| | | |
|---|---|---|
| AJCC | Sex | mean |

| STAGE | n= | male | female | (range) |
|---|---|---|---|---|
| IIB | 2 | 1 | 1 | 76 (75-76) |
| III | 1 | 1 | 0 | 68 |

### Sample Treatment

Blood was collected by venipuncture into Vacutainer® gel tubes without anticoagulant (serum samples) or with dipotassium-EDTA (plasma samples). Blood samples were allowed to cloth for 30 minutes and centrifuged at 1200×g for 10 minutes at 4° C. Serum and plasma samples were aliquoted and stored at −80° C. All samples were collected and processed using the same protocol.

Serum and plasma samples obtained from cancer patients and control population were treated based on a method reported in Gómez-Gómez et al (Gómez-Gómez et al, Journal of Pharmaceutical and Biomedical Analysis. 2022 January; 208, 114450). First, proteins were precipitated by adding 40 UL of cold methanol to 10 μL of plasma (5-fold plasma dilution). Then, 25 UL of the diluted sample were mixed with 30 UL of internal standard working solution consisting on a methanolic mixture containing octanoate-13C4, β-hydroxybutyrate-d4, Lactate-d3 and pyruvate-13C3 all at 0.5 g/mL. Targeted metabolites were derivatized using 500 UL of o-benzyl hydroxylamine and 500 μL of N-(3-Dimethylaminopropyl)-N′-ethylcarbodiimide hydrochloride at 1 M) during 1 h at room temperature. Derivatization reaction was stopped by addition of 1 mL of milliQ water. Analytes were extracted by a liquid-liquid extraction after addition of 4 mL of ethyl acetate and vortexing at maximum power during 1 minute. After centrifugation (5 minutes, 3800 rpm), the organic layer was separated and dried under a nitrogen stream in a water bath at 40° C. The extracts were reconstituted in 300 μL of water: methanol (1/1). Finally, 10 UL were injected into the liquid chromatography-tandem mass spectrometry (LC-MS/MS) system.

### LC-MS/MS

The LC-MS/MS system was an Acquity UPLC system (Waters Associated, Milford, MA, USA) coupled to a triple quadrupole (Xevo TQ-S) mass spectrometer (Waters Associated) provided with an orthogonal Z-spray-electrospray interface (ESI).

The LC chromatography separation was performed using an Acquity BEH C18 column (100 mm×2.1 mm i.d., 1.7 μm) (Waters Associates) with a flow rate of 0.3 mL/min at 55° C. The selected mobile phases were water-ammonium formate (1 mM)-formic acid (0.01%) as mobile phase A and methanol-ammonium formate (1 mM)-formic acid (0.01%) as mobile phase B.

The MS/MS detection was achieved by using the positive ionization mode. The MS used argon as collision gas and nitrogen as drying and nebulizing gas. The desolvation gas flow was set at 1200 L/h and the cone gas at 50 L/h. A capillary voltage of 3 kV was selected for the MS detection. Nitrogen desolvation temperature and source temperature were set to 600° C. and 150° C., respectively.

A targeted LC-MS/MS method was developed for the quantification of the metabolites of interest. The approach used a 8.75 minutes chromatographic gradient in which the percentage of the methanolic mobile phase (B) linearly increased as follows: 1 min, 20%; 2 min, 30%; 2.01 min, 35%; 2.75 min, 45%; 2.76 min, 65%; 6.75 min, 85%; 6.76 min, 95%; 7.75 min, 95%; 7.76 min, 20%; 8.75 min, 20%. Determination of the metabolites was performed by a selected reaction monitoring method including two ion transitions per metabolite. The main characteristics of the method are shown in Table 8. Quantification was performed by external calibration comparing the response (area of the analyte/area of the internal standard) of the metabolites in the samples with the response obtained for calibration standards with known concentrations.

TABLE 8
SRM conditions for the targeted determination of metabolites

| | Retention | Precursor | Cone | Collision | Production |
|---|---|---|---|---|---|
| Metabolite | time (min) | ion (m/z) | voltage(V) | energy (V) | (m/z) |
| Octanoate | 5.52 | 250 | 20 | 20 | 124^{a} |
| | | | 20 | 10 | 91 |
| Decanoate | 6.65 | 278 | 20 | 20 | 124^{a} |
| | | | 20 | 10 | 91 |
| Acetoacetate | 4.89 | 313 | 20 | 10 | 181^{a} |
| | | | 20 | 20 | 91 |
| β-hydroxybutyrate | 3.45 | 210 | 20 | 10 | 124^{a} |
| | | | 20 | 20 | 91 |
| 3-oxopropanoate | 4.67 | 299 | 20 | 10 | 176^{a} |
| | | | 20 | 20 | 91 |
| Gluconate | 1.96 | 302 | 20 | 10 | 124^{a} |
| | | | 20 | 20 | 91 |
| α-ketobutyrate | 5.51 | 313 | 20 | 10 | 190^{a} |
| | | | 20 | 20 | 91 |
| Octanoate-¹³C₄ | 5.52 | 254 | 20 | 20 | 124^{a} |
| | | | 20 | 10 | 91 |
| β-hydroxybutyrate-d₄ | 3.44 | 214 | 20 | 10 | 124^{a} |
| | | | 20 | 20 | 91 |
| Lactate-d₃ | 3.26 | 199 | 20 | 5 | 124^{a} |
| | | | 20 | 10 | 91 |
| Pyruvate-¹³C₃ | 5.16 | 302 | 20 | 10 | 181^{a} |
| | | | 20 | 20 | 91 |

### Statistics

The suitability of the selected metabolite signature for discerning between cancer and controls for the different tumors was evaluated by performing a receiver operating characteristic (ROC) and by calculating the corresponding area under the curve (AUC). ROC curves and AUC values were performed using SPSS software (v 18.0; IBM, Armonk, New York, NY, USA).

In order to obtain a population threshold for each selected biomarker, we accurately quantified the concentrations for each metabolite in an extended Control Cohort comprising 134 healthy controls (HC) recruited at Hospital del Mar (Barcelona, Spain) (n=95), Hospital Vall d′Hebrón (Barcelona, Spain) (n=30) and Hospital Trueta (Girona, Spain) (n=9). This cohort was used to establish both reference and threshold values. Population threshold for each marker of the signature was calculated considering the following equation:

Threshold>(controls concentration average)+3×(controls standard deviation).

These thresholds would account for at least 99.7% of the population. Thus, individuals with results below the threshold for all metabolites can be considered negative and results above the threshold for any of the biomarkers of the signature can result in a positive case.

TABLE 9
Concentrations in controls and established
threshold for each metabolite

| | Controls (n = 134) | | |
|---|---|---|---|
| | Mean | Range* | Threshold |
| Biomarker | ng/mL | | |
| Octanoate | 119 | 2.3-1158 | 2000 |
| Decanoate | 274 | 44-1594 | 3000 |
| β-OH-Butyrate | 7853 | 1997-30888 | 50000 |
| Acetoacetate | 2577 | 139-11235 | 12500 |
| 3-oxopropanoate | 19 | 6.1-67 | 250 |
| Gluconate | 1104 | 42-3192 | 5000 |
| α-Ketobutyrate | 166 | 20-590 | 700 |
| Octanoate + Decanoate | 393 | 82-2184 | 4000 |
| β-OH-Butyrate + Acetoacetate | 10430 | 2515-40567 | 55000 |

*Results of range expressed as “minimum-maximum”

### 1.2. Results

1.2.1. Pancreatic cancer

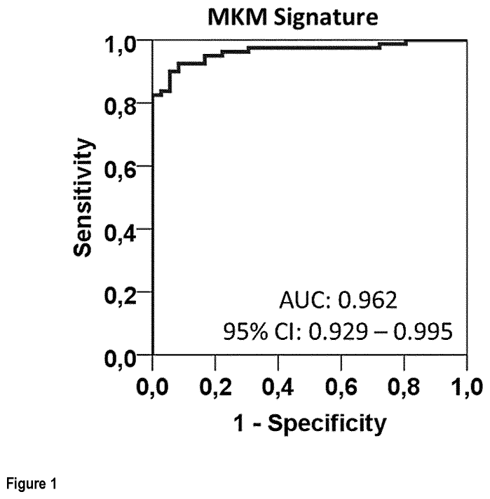

The MKM metabolic signature containing the biomarkers 3-oxopropanoate, beta-hydroxybutyrate and decanoate was evaluated in a cohort of pancreatic cancer patients (cases, N=80) and non-oncologic patients (controls, which included both healthy subjects and chronic pancreatitis patients, N=36). The MKM signature was able to differentiate cases from controls (AUC=0.962) as can be seen in FIG. 1.

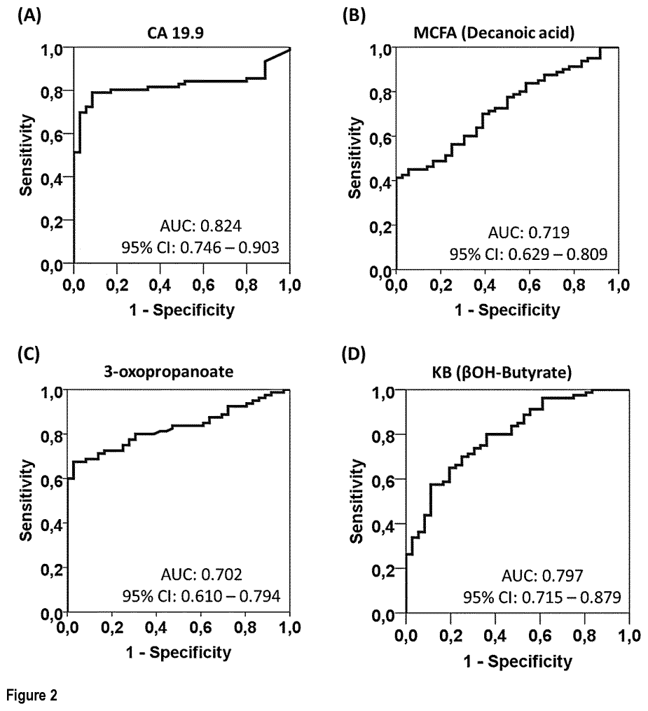

The predictive potential of the MKM signature for pancreatic cancer diagnosis was higher than that of CA19.9 (FIG. 2A) and by the individual components of the signature (FIGS. 2B-2D).

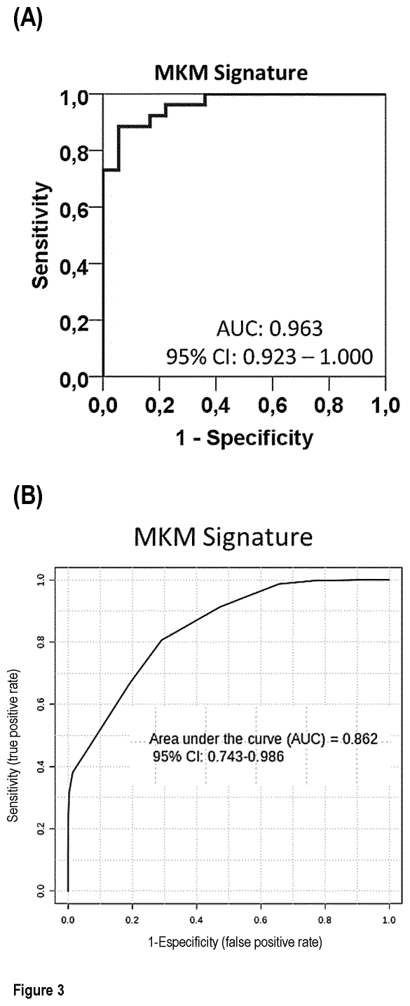

In order to check the potential of the MKM signature for the early diagnosis of pancreatic cancer, we selected from the pancreatic cancer cohort those patients diagnosed in stages I and IIA. The early stage cohort of pancreatic cancer consisted on patients (cases, N=26) and non-oncologic patients (controls, which included both healthy subjects and chronic pancreatitis patients, N=36). The MKM signature was able to differentiate cases from controls in a similar way than when using the whole cohort (AUC=0.963) as can be seen in FIG. 3.

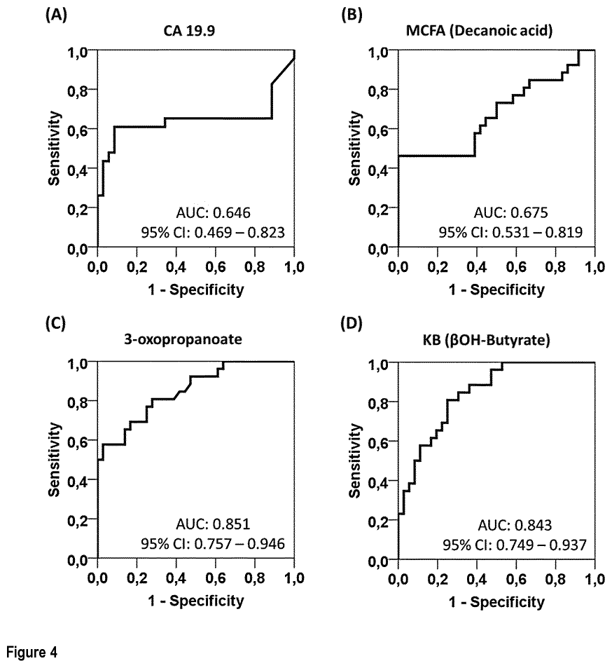

The potential of the MKM signature for early pancreatic cancer diagnosis was higher than the obtained by CA19.9 (FIG. 4A) and by the individual components of the signature (FIGS. 4B-4D).

An extended MKM metabolic signature was also evaluated. This extended MKM signature contained, in addition to 3-oxopropanoate, beta-hydroxybutyrate and decanoate, the following metabolites: octanoate, acetoacetate, gluconate and alpha-ketobutyrate. Results for the 7-biomarker signature are shown in FIGS. 5-7. It may be seen that the addition of selected biomarkers as disclosed above to the basic MKM signature further improved the discriminating capacity of the diagnostic method of the invention.

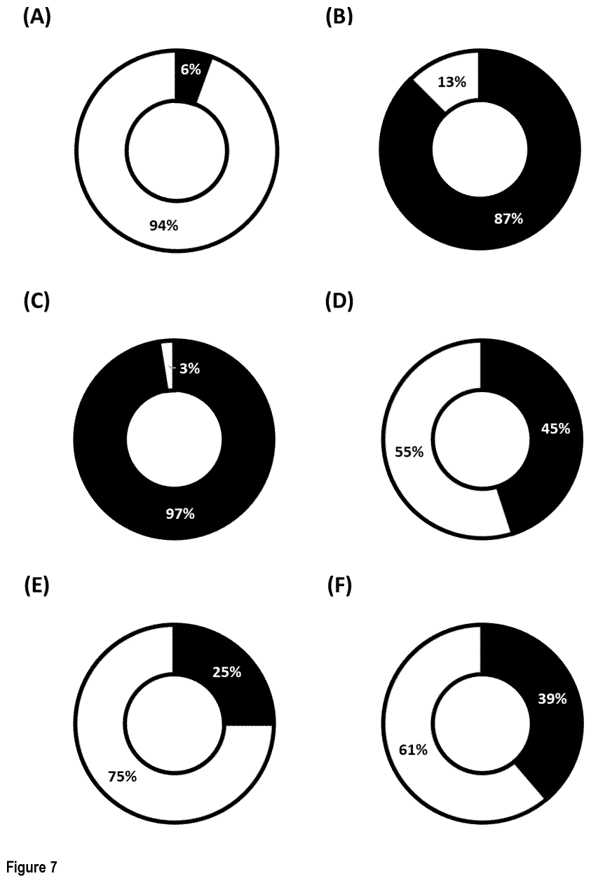

The percentage of patients included in the whole pancreatic cancer cohort who had an increased plasma concentration of at least one of the 7 biomarkers was calculated. The same calculation was performed for a group of non-oncologic patients which included both healthy and chronic pancreatitis patients. We also calculated the percentage of patients included in the whole pancreatic cancer cohort who tested positive for plasma CA19.9 and also had increased plasma concentration of at least one of the 7 biomarkers. It was found that 87% of the blood samples from pancreatic cancer patients included in the study had increased levels of at least one of the metabolites (FIG. 7B), while only two of the non-oncologic subjects had at least one of the metabolites (6%) (FIG. 7A). It was also found that 97% of pancreatic cancer patients included in the study had increased levels of at least one of the metabolites in addition to CA19.9 (FIG. 7C).

Heatmaps were also computed to evaluate the occurrence of biomarker's overexpression in an extended cohort of pancreatic cancer and control patients, as well as chronic pancreatitis and IPMN patients (FIG. 15). It is clear from the heatmaps that most early stage pancreatic cancer patients had at least one overexpressed biomarker from the 7 biomarker panel. In contrast, none of the control patients and very few chronic pancreatitis patients and IPMN patients had overexpressed biomarkers.

### 1.2.2. Gastric Cancer

The MKM signature was also evaluated in a cohort of gastric cancer patients (cases, N=127) and non-oncologic patients (controls, N=52). The MKM signature was able to differentiate cases from controls (AUC=0.760) as can be seen in FIG. 8A.

The use of the seven markers signature improved the results obtaining an AUC=0.860 as can be seen in FIG. 8B

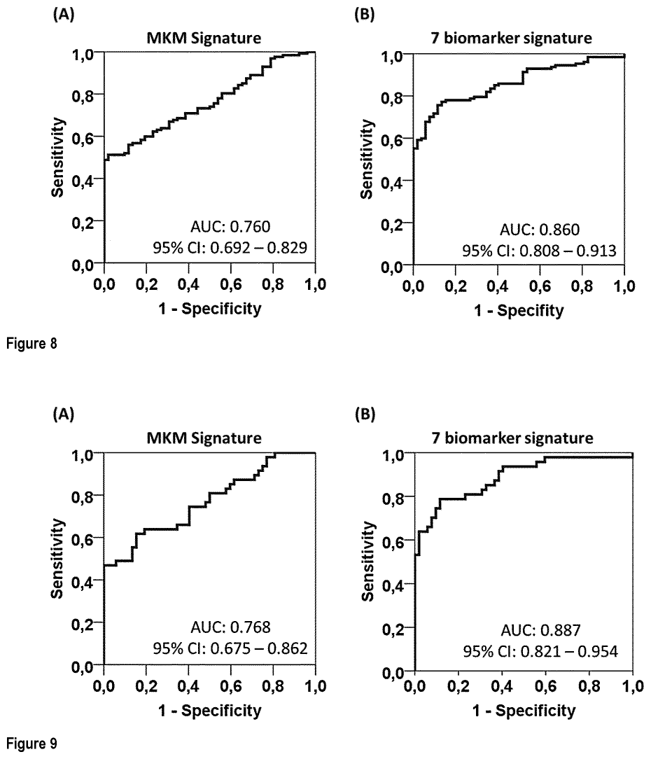

Similar results were obtained for early diagnosis i.e., when only considering those patients diagnosed in stages 0, I and II (N=63). The MKM signature was able to differentiate cases from controls in a similar way than when using the whole cohort (AUC=0.768) as can be seen in FIG. 9A.

The use of the seven markers signature improved the results obtaining an AUC=0.887 as can be seen in FIG. 9B.

### 1.2.3. Esophageal Cancer

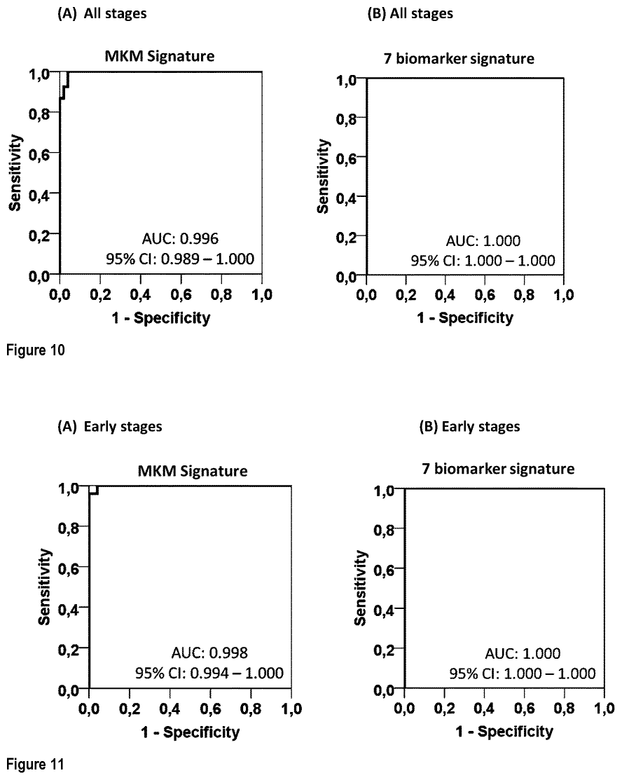

The MKM signature was also evaluated in a cohort of esophageal cancer patients (cases, N=52) and non-oncologic patients (controls, N=50). The MKM signature was able to differentiate cases from controls (AUC=0.996) as can be seen in FIG. 10A. The use of the seven markers signature improved the results obtaining an AUC=1.000 as can be seen in FIG. 10B. Similar results were obtained for early diagnosis i.e., when only considering those patients diagnosed in stages 0, I and II (N=24). The MKM signature was able to differentiate cases from controls in a similar way than when using the whole cohort (AUC=0.998) as can be seen in FIG. 11A. The use of the seven markers signature improved the results obtaining an AUC=1.000 as can be seen in FIG. 11B.

### 1.2.4. Gynecological Cancer

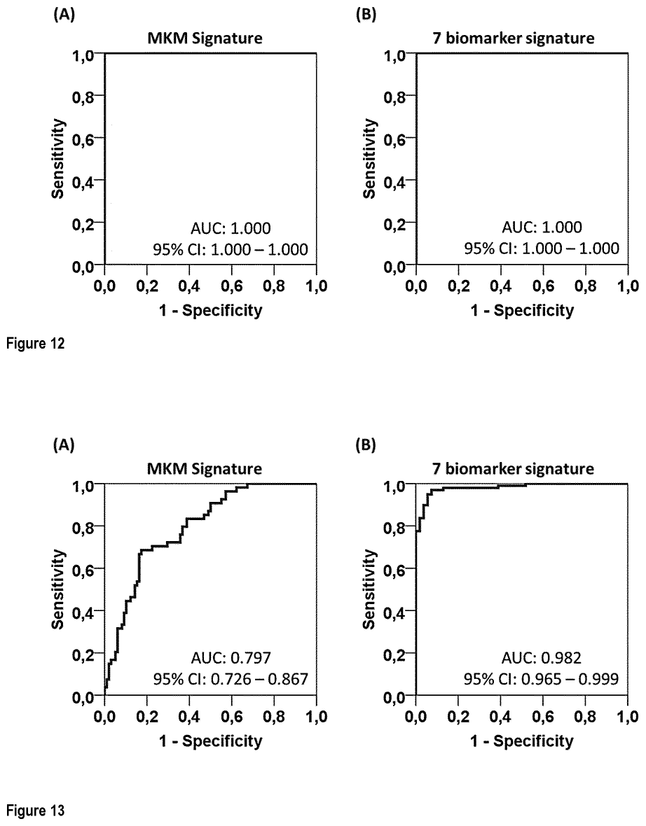

The MKM signature was also evaluated in a cohort of gynecological cancer patients (cases, N=9) and non-oncologic patients (controls, N=6). The MKM signature was able to differentiate cases from controls (AUC=1.000) as can be seen in FIG. 12A. The use of the seven markers signature also yielded an AUC=1.000 as can be seen in FIG. 12B

### 1.2.5. Lung Cancer

The MKM signature was also evaluated in a cohort of small cell lung cancer patients (SCLC, cases, N=98) and non-oncologic patients (controls, N=50). The MKM signature was able to differentiate cases from controls (AUC=0.797) as can be seen in FIG. 13A. The use of the seven markers signature improved the results obtaining an AUC=0.982 as can be seen in FIG. 13B.

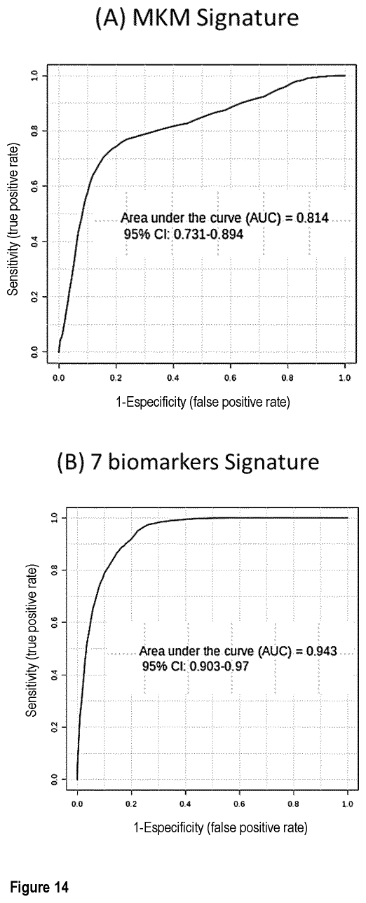

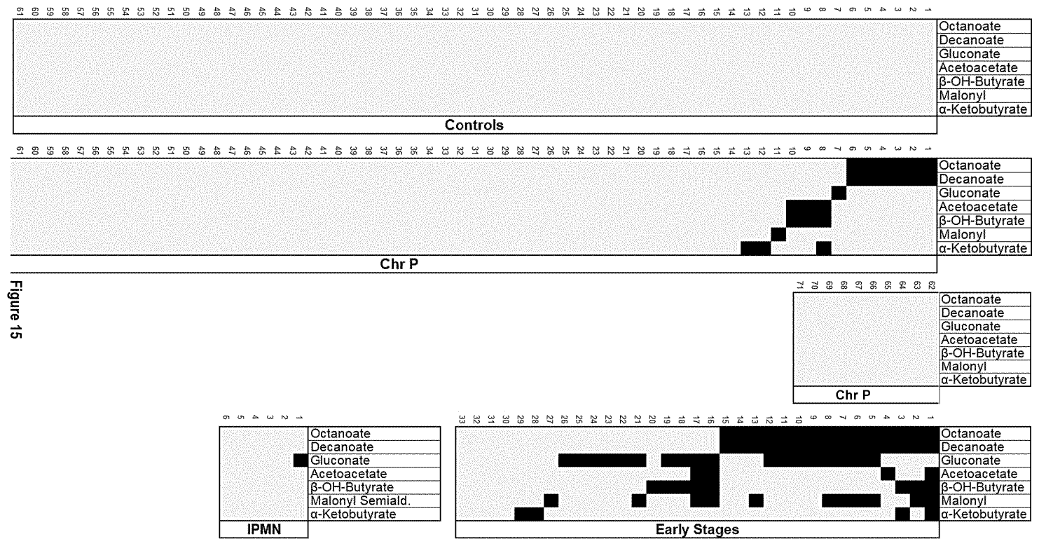

The MKM signature was also evaluated in a cohort of non-small cell lung cancer patients (NSCLC, cases, N=92) and non-oncologic patients (controls, N=50). The MKM signature was able to differentiate cases from controls (AUC=0.814) as can be seen in FIG. 14A. The use of the seven markers signature improved the results obtaining an AUC=0.943 as can be seen in FIG. 14B

### 1.2.6. Cholangiocarcinoma

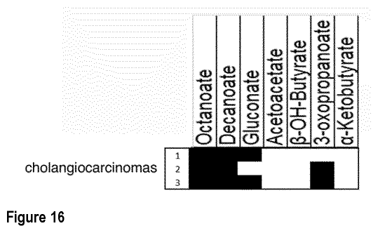

The results obtained for the 7-metabolite signature in the Cholangiocarcinoma cohort are shown in the heatmap of FIG. 16. All evaluated patients had at least one overexpressed biomarker from this signature.

### CITATION LIST

Burtis C. A. et al., 2008, Chapter 14, section “Statistical Treatment of Reference Values”
Gillen S, Schuster T, Büschenfelde C M Zum, et al: Preoperative/neoadjuvant therapy in pancreatic cancer: A systematic review and meta-analysis of response and resection percentages. PLOS Medicine 7, 2010. PMID: 20422030 PMCID: PMC2857873.
Gómez-Gómez et al. Determination of up to twenty carboxylic acid containing compounds in clinically relevant matrices by o-benzylhydroxylamine derivatization and liquid chromatography-tandem mass spectrometry. Journal of Pharmaceutical and Biomedical Analysis 2022 Jan. 20; 208:114450. PMID: 34798391https://doi.org/10.1016/j.jpba. 2021.114450
Kenner B, Chari S T, Kelsen D, et al. Artificial Intelligence and Early Detection of Pancreatic Cancer: 2020 Summative Review. Pancreas. 2021; 50 (3): 251-279. PMID: 33835956 PMCID: PMC8041569
Li R, Kenyon G L. A spectrophotometric determination of alpha-dicarbonyl compounds and its application to the enzymatic formation of alpha-ketobutyrate. Anal Biochem. 1995 Sep. 1; 230 (1): 37-40 PMID: 8585627
Luo G, Jin K, Deng S, Cheng H, Fan Z, Gong Y, Qian Y, Huang Q, Ni Q, Liu C, Yu X. Roles of CA19-9 in pancreatic cancer: Biomarker, predictor and promoter. Biochim Biophys Acta Rev Cancer. 2021 April; 1875 (2): 188409. doi: 10.1016/j.bbcan.2020.188409. Epub 2020 Aug. 19. PMID: 32827580
Mellby L D, Nyberg A P, Johansen J S, Wingren C, Nordestgaard B G, Bojesen S E, Mitchell B L, Sheppard B C, Sears R C, Borrebaeck C A K. Serum Biomarker Signature-Based Liquid Biopsy for Diagnosis of Early-Stage Pancreatic Cancer. J Clin Oncol. 2018 Oct. 1; 36 (28): 2887-2894. doi: 10.1200/JCO.2017.77.6658. Epub 2018 Aug. 14. PMID: 30106639; PMCID: PMC6161836.
Park W, Chawla A, O'Reilly E M. Pancreatic Cancer: A Review. JAMA-Journal of the American Medical Association 326:851-862, 2021. PMID: 34547082
Rahib L, Smith B D, Aizenberg R, et al. Projecting cancer incidence and deaths to 2030: The unexpected burden of thyroid, liver, and pancreas cancers in the United States. Cancer Research 74:2913-2921, 2014. PMID: 24840647
Siegel R L, Miller K D, Fuchs H E, Jemal A. Cancer statistics, 2022. CA Cancer J Clin. 2022 January; 72 (1): 7-33. doi: 10.3322/caac.21708. Epub 2022 Jan. 12. PMID: 35020204.

1-15. (canceled)

16. A method for treating a subject suffering from cancer, said method comprising diagnosing that the subject suffers from cancer and subsequently administering an appropriate therapeutic regime for cancer, wherein the diagnosing comprises the steps of:
(i) determining, in a sample obtained from the subject, the concentration of (a) 3-oxopropanoate, and at least one further metabolite selected from (b) a ketone body selected from beta-hydroxybutyrate, acetoacetate, and combinations thereof, and (c) a medium chain fatty acid selected from octanoate, decanoate, and combinations thereof,
(ii) comparing the concentration of each of the metabolites determined in (i) with a corresponding reference value, and
(iii) diagnosing that the patient has cancer by determining that the concentration of at least one of (a), (b) or (c) is increased as compared to the corresponding reference value, wherein the reference value for each metabolite is the concentration of said metabolite determined in a biological sample obtained from a non-oncologic subject or group of subjects.

17. The method according to claim 16, wherein the therapeutic regime is selected from the group consisting of surgical resection, radiotherapy, chemotherapy, immunotherapy, hormonal therapy, hyperthermia, photodynamic therapy, stem cell transplant, and combinations thereof.

18. The method according to claim 16, wherein step (i) comprises determining (a), (b), and (c).

19. The method according to claim 18, wherein step (i) comprises determining 3-oxopropanoate, beta-hydroxybutyrate, acetoacetate, octanoate, and decanoate.

20. The method according to claim 16, wherein step (i) further comprises determining the concentration of a metabolite selected from (d) alpha-ketobutyrate, (e) gluconate, and combinations thereof, and step (iii) comprises diagnosing that the patient has cancer by determining that the concentration of at least one of the metabolites determined in (i) is increased as compared to the corresponding reference value.

21. The method according to claim 20, wherein step (i) comprises determining 3-oxopropanoate, beta-hydroxybutyrate, acetoacetate, octanoate, decanoate, alpha-ketobutyrate, and gluconate, and step (iii) comprises diagnosing that the patient has cancer by determining that the concentration of at least one of the metabolites determined in (i) is increased as compared to the corresponding reference value.

22. The method according to claim 16, wherein the increase is at least 10-fold.

23. The method according to claim 16, wherein the subject is diagnosed as having early stage cancer in stages IA, IB or IIA.

24. The method according to claim 16, wherein the method is characterized by a receiver operating characteristic area under the curve value in the range from 0.800 to 1.000.

25. The method according to claim 16, that is for diagnosing a solid tumor.

26. The method according to claim 25, wherein the cancer is selected from the group consisting of pancreatic cancer, gastric cancer, esophageal cancer, gynecological cancer, liver cancer, breast cancer, colorectal cancer, and lung cancer, in particular pancreatic cancer.

27. The method according to claim 16, wherein the sample is selected from blood, plasma and serum.

28. The method according to claim 16, wherein the concentration of metabolites is determined by a method selected from the group consisting of liquid chromatography with tandem mass spectrometry, enzymatic analysis, colorimetric analysis, fluorometric analysis, luminometric analysis and turbidimetric analysis.

29. The method according to claim 28, wherein the concentration of metabolites is determined by liquid chromatography with tandem mass spectrometry.

30. A kit of parts comprising:
(i) means for determining the concentration of (a) 3-oxopropanoate, and at least one other metabolite selected from (b) a ketone body selected from beta-hydroxybutyrate, acetoacetate and combinations thereof, and (c) a medium chain fatty acid selected from octanoate, decanoate and combinations thereof, and optionally means for determining the concentration of a further metabolite selected from (d) alpha-ketobutyrate, (e) gluconate, and combinations thereof,
(ii) optionally, reference values for the above metabolites, and
(iii) instructions for diagnosing cancer.

31. The kit according to claim 30, comprising means for determining the concentration of (a), (b), and (c).

32. The kit according to claim 31, comprising means for determining the concentration of 3-oxopropanoate, beta-hydroxybutyrate, acetoacetate, octanoate, and decanoate.

33. The kit according to claim 32, comprising means for determining the concentration of 3-oxopropanoate, beta-hydroxybutyrate, acetoacetate, octanoate, decanoate, alpha-ketobutyrate, and gluconate.

34. A method for treating a subject suffering from cancer, said method comprising diagnosing that the subject suffers from cancer, wherein the diagnosing comprises the steps of:
(i) determining, in a sample obtained from the subject, the concentration of (a) 3-oxopropanoate, and at least one further metabolite selected from (b) a ketone body selected from beta-hydroxybutyrate, acetoacetate, and combinations thereof, and (c) a medium chain fatty acid selected from octanoate, decanoate, and combinations thereof,
(ii) comparing the concentration of each of the metabolites determined in (i) with a corresponding reference value, and
(iii) diagnosing that the patient has cancer by determining that the concentration of at least one of (a), (b) or (c) is increased as compared to the corresponding reference value, wherein the reference value for each metabolite is the concentration of said metabolite determined in a biological sample obtained from a non-oncologic subject or group of subjects.
